(19) United States
(12) Patent Application Publication
(10) Pub. No.: US 2024/0075303 A1
(43) Pub. Date: Mar. 7, 2024

(54) SYSTEMS AND METHODS FOR DIRECT VISUALIZATION OF A TISSUE LOCATION, SUCH AS AN ENDOCARDIAL LOCATION

(71) Applicant: EBR Systems, Inc., Sunnyvale, CA (US)

(72) Inventor: Nathaniel Parker Willis, Sunnyvale, CA (US)

(21) Appl. No.: 18/240,939

(22) Filed: Aug. 31, 2023

Related U.S. Application Data
(60) Provisional application No. 63/403,277, filed on Sep. 1, 2022.

(51) Int. Cl. A61N 1/372; G06T 1/00

(52) U.S. Cl. CPC A61N 1/372; G06T 1/0007; G06T 2207/30004

(57) ABSTRACT

The present technology is generally directed to delivery systems for medical implants, such as stimulation assemblies for stimulating tissue of a human patent, and associated methods. In some embodiments, a delivery system includes (i) an elongate sheath having a distal portion with a distal opening, (ii) an attachment mechanism positioned within the elongate sheath and configured to be releasably coupled to an electrical stimulation implant, and (iii) an elongate optical component movably positioned within the elongate sheath. The optical component is configured to capture image data, and is movable from a first configuration to a second configuration. In the first configuration, the optical component is positioned proximally of the implant. In the second configuration, the optical component is positioned at least partially adjacent to the implant to capture image data proximate the distal opening, thereby facilitating direct visualization of a target location for implantation of the implant.

### CROSS-REFERENCE TO RELATED APPLICATION

This application claims the benefit of U.S. Provisional Patent Application No. 63/403,277, filed Sep. 1, 2022, and titled “SYSTEMS AND METHODS FOR DIRECT VISUALIZATION OF A TISSUE LOCATION, SUCH AS AN ENDOCARDIAL LOCATION,” which is incorporated herein by reference in its entirety.

### TECHNICAL FIELD

The present technology generally relates to systems for stimulating tissue and, more particularly, to delivery systems and associated methods for visualizing a tissue location, such as an endocardial tissue location, during delivery of an electrical stimulation implant to the tissue location.

### BACKGROUND

Electrical stimulation of body tissue is used throughout medicine for treatment of both chronic and acute conditions. Among many examples, peripheral muscle stimulation is reported to accelerate healing of strains and tears, bone stimulation is likewise indicated to increase the rate of bone regrowth/repair in fractures, and nerve stimulation is used to alleviate chronic pain. Further there is encouraging research in the use of electrical stimulation to treat a variety of nerve and brain conditions, such as essential tremor, Parkinson's disease, migraine headaches, functional deficits due to stroke, and epileptic seizures.

Cardiac pacemakers are examples of commonly implanted device utilizing electrical stimulation to stimulate cardiac and other tissues. A pacemaker is a battery-powered electronic device implanted under the skin, connected to the heart by an insulated metal lead wire with a tip electrode. Pacemakers were initially developed for and are most commonly used to treat slow heart rates (bradycardia), which may result from a number of conditions. More recently, advancements in pacemaker complexity, and associated sensing and pacing algorithms have allowed progress in using pacemakers for the treatment of other conditions, notably heart failure (HF) and fast heart rhythms (tachyarrhythmia/tachycardia).

Electrical energy sources connected to electrode/lead wire systems have typically been used to stimulate tissue within the body. The use of lead wires is associated with significant problems such as complications due to infection, lead failure, and electrode/lead dislodgement. The requirement for leads to accomplish stimulation also limits the number of accessible locations in the body. The requirement for leads has also limited the ability to stimulate at multiple sites (multisite stimulation).

Accordingly, some systems utilize wireless (e.g., leadless) stimulation electrodes that can be implanted in tissue and receive energy from a separate source or self-contained source (e.g., a battery) to deliver electrical stimulation to the tissue. Wireless stimulation electrodes are typically delivered via catheter-based delivery systems. One difficulty associated with implanting a wireless stimulation electrode is visualizing a target tissue location before implantation of the wireless stimulation electrode at the target tissue location. Typical delivery methods include the use of fluoroscopic and/or echocardiographic techniques to provide visual feedback of the position and location of the delivery catheter relative to the target tissue location.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic diagram of a tissue stimulation system configured in accordance with embodiments of the present technology.

FIGS. 2A and 2B are enlarged side views of a distal portion of a delivery system configured to facilitate delivery of an implantable medical device to tissue of a patient in accordance with embodiments of the present technology.

FIGS. 3A-3D are enlarged side views of the distal portion of the delivery system of FIGS. 2A and 2B during a procedure to implant the implantable medical device in the tissue of the patient in accordance with embodiments of the present technology.

FIGS. 4A-4C are enlarged side views of a distal portion of a delivery system configured to facilitate delivery of an implantable medical device to tissue of a patient in accordance with additional embodiments of the present technology.

FIG. 5A is an enlarged side cross-sectional view of a portion of a receiver-stimulator and an advancement and attachment mechanism shown in FIG. 2A in accordance with embodiments of the present technology.

FIG. 5B is an enlarged perspective view of a distal portion of an intermediate member of the advancement and attachment mechanism of FIG. 5A in accordance with embodiments of the present technology.

### DETAILED DESCRIPTION

Aspects of the present disclosure are directed to systems and methods for delivering medical implants, such as electrical stimulation implants, to tissue (e.g., cardiac tissue) of a patient. In several of the embodiments described below, for example, a delivery system includes (i) an elongate sheath having a distal portion with a distal opening, (ii) an attachment mechanism positioned within the sheath and configured to be releasably coupled to an electrical stimulation implant (“the implant”), and (iii) an elongate optical component movably positioned within the sheath. The optical component is configured to capture image data, and is movable from a first configuration to a second configuration. In the first configuration, the optical component is positioned proximal to the implant. In the second configuration, the optical component is positioned adjacent to at least a portion of the implant to capture image data at and/or proximate to the distal opening, thereby providing direct visualization of a target location before and/or during implantation of the implant. In some embodiments, the distal portion of the sheath is formed from a compliant material that can expand when the optical component moves to the second configuration.

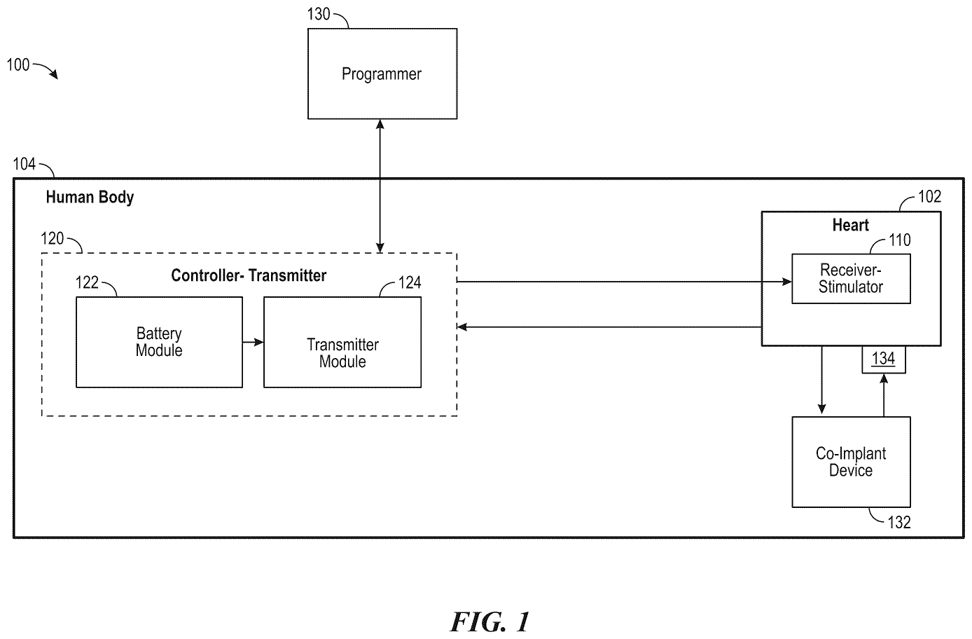

Specific details of several embodiments of the present technology are described herein with reference to FIGS. 1-5B. The present technology, however, can be practiced without some of these specific details. In some instances, well-known structures and techniques often associated with wireless (e.g., leadless) tissue stimulation systems, cardiac pacing, electronic circuitry, acoustic and radiofrequency transmission and receipt, delivery systems and catheters, and the like, have not been shown in detail so as not to obscure the present technology. Moreover, although many of the embodiments are described below with respect to systems and methods for endocardial tissue pacing, other applications and other embodiments in addition to those described herein are within the scope of the technology. For example, one of ordinary skill in the art will understand that one or more aspects of the present technology are applicable to other implantable devices configured to treat other areas of the human body.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

With regard to the terms “distal” and “proximal” within this description, unless otherwise specified, the terms can reference a relative position of the portions of a catheter subsystem with reference to an operator and/or a location in the vasculature. For example, “distal” may refer to a position or movement in a direction further away from the operator, and “proximal” may refer to a position or movement in a direction closer to the operator. Also, as used herein, the designations “rearward,” “forward,” “upward,” “downward,” and the like are not meant to limit the referenced component to a specific orientation. It will be appreciated that such designations refer to the orientation of the referenced component as illustrated in the drawings; the systems of the present technology can be used in any orientation suitable to the user.

To the extent any materials incorporated herein by reference conflict with the present disclosure, the present disclosure controls. As used herein, the terms about, substantially, approximately, and the like, mean within plus or minus 10% of the stated value unless otherwise noted.

FIG. 1 is a schematic diagram of a tissue stimulation system 100 (“system 100”) configured in accordance with embodiments of the present technology. In the illustrated embodiment, the system 100 is configured to stimulate a heart 102 within a body 104 of a human patient. The system 100 can include one or more receiver-stimulators 110 (one shown in FIG. 1; which can also be referred to as stimulators, stimulation assemblies, ultrasound receivers, stimulating electrodes, stimulation electrodes, pacing electrodes, acoustic receivers, and the like) in operable communication (e.g., wireless, acoustic, and/or radio communication) with a controller-transmitter 120 (which can also be referred to as an ultrasound transmitter, a pulse generator, an acoustic transmitter, and the like). The controller-transmitter 120 can include a battery module 122 and a transmitter module 124 operably coupled to and powered via the battery module 122. In some embodiments, both the receiver-stimulator 110 and the controller-transmitter 120 are configured to be implanted within the body 104 of the human patient. For example, the receiver-stimulator 110 can be implanted at and/or proximate the heart 102 (e.g., in the left ventricle, the right ventricle, or proximate area) for delivering stimulation pulses to the heart 102, while the controller-transmitter 120 can be positioned at another location remote from the heart 102 (e.g., in the chest area). In a particular embodiment, the receiver-stimulator 110 is positioned within the left ventricle and configured to stimulate endocardial tissue of the septal wall. The transmitter module 124 of the controller-transmitter 120 can direct energy (e.g., acoustic energy, ultrasound energy) toward the receiver-stimulator 110, which can receive the energy and deliver one or more electrical pulses (e.g., stimulation pulses, pacing pulses) to the heart 102.

In some embodiments, the system 100 can further include a programmer 130 in operable communication with the controller-transmitter 120. The programmer 130 can be positioned outside the body 104 and can be operable to program various parameters of the controller-transmitter 120 and/or to receive diagnostic information from the controller-transmitter 120. In some embodiments, the system 100 further includes a co-implant device 132 (e.g., an implantable cardioverter defibrillator (ICD) or pacemaker) coupled to pacing leads 134 for delivering stimulation pulses to one or more portions of the heart 102 other than the area stimulated by the receiver-stimulator 110. In other embodiments, the co-implant device 132 can be a leadless pacemaker which is implanted directly into the heart 102 to eliminate the need for separate pacing leads 134. The co-implant device 132 and the controller-transmitter 120 can operate in tandem and deliver stimulation signals to the heart 102 to cause a synchronized heartbeat. In some embodiments, the controller-transmitter 120 receives signals (e.g., electrocardiogram signals) from the heart 102 to determine information related to the heart 102, such as a heart rate, heart rhythm, including the output of the pacing leads 134 located in the heart 102. In some embodiments, the controller-transmitter 120 alternatively or additionally receives information (e.g., diagnostic signals) from the receiver-stimulator 110. The received signals can be used to adjust the ultrasound energy signals delivered to the receiver-stimulator 110.

The receiver-stimulator 110, the controller-transmitter 120, and/or the programmer 130 can include a machine-readable (e.g., computer-readable) or controller-readable medium containing instructions for generating, transmitting, and/or receiving suitable signals (e.g., stimulation signals, diagnostic signals). The receiver-stimulator 110, the controller-transmitter 120, and/or the programmer 130 can include one or more processor(s), memory unit(s), and/or input/output device(s). Accordingly, the process of providing stimulation signals and/or executing other associated functions can be performed by computer-executable instructions contained by, on, or in computer-readable media located at the receiver-stimulator 110, the controller-transmitter 120, and/or the programmer 130. Further, the receiver-stimulator 110, the controller-transmitter 120, and/or the programmer 130 can include dedicated hardware, firmware, and/or software for executing computer-executable instructions that, when executed, perform any one or more methods, processes, and/or sub-processes described herein. The dedicated hardware, firmware, and/or software also serve as “means for” performing the methods, processes, and/or sub-processes described herein.

In some embodiments, the system 100 can include several features generally similar or identical to those of the leadless tissue stimulation systems disclosed in (i) U.S. Pat. No. 7,610,092, filed Dec. 21, 2005, and titled “LEADLESS TISSUE STIMULATION SYSTEMS AND METHODS,” (ii) U.S. Pat. No. 8,315,701, filed Sep. 4, 2009, and titled “LEADLESS TISSUE STIMULATION SYSTEMS AND METHODS,” and/or (iii) U.S. Pat. No. 8,718,773, filed May 23, 2007, and titled “OPTIMIZING ENERGY TRANSMISSION IN A LEADLESS TISSUE STIMULATION SYSTEM.”

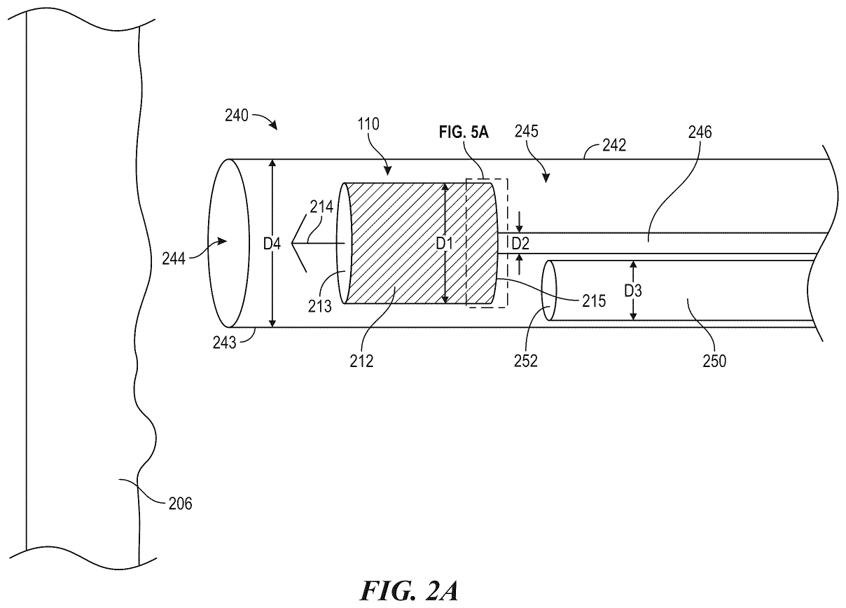

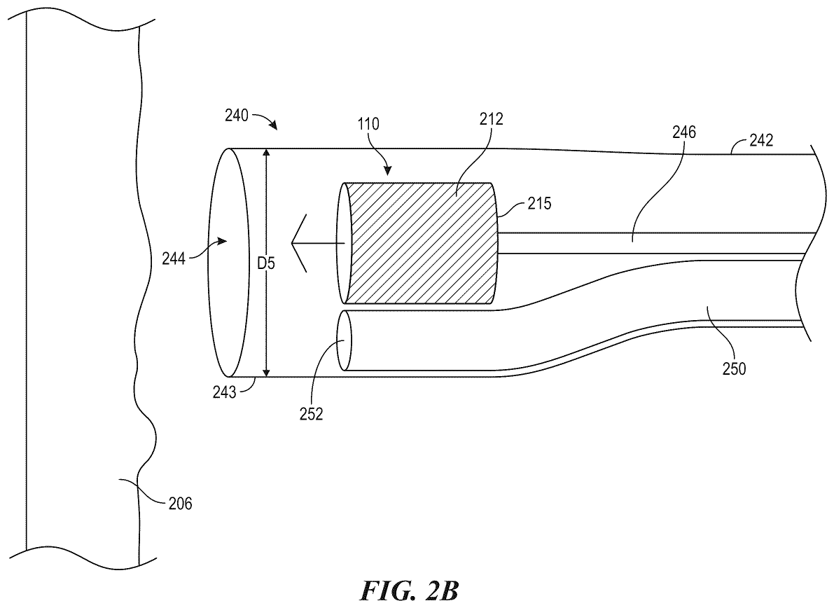

FIGS. 2A and 2B are enlarged side views of a distal portion of a delivery system 240 configured to facilitate delivery of an implantable medical device to tissue 206 of a patient in accordance with embodiments of the present technology. The delivery system 240 is in a first position (also referred to as a “first configuration” or “first state”; e.g., a delivery configuration, an advancement configuration) in FIG. 2A, and in a second position (also referred to as a “second configuration” or “second state”; e.g., a visualization configuration, an implantation configuration) in FIG. 2B. In some embodiments, the medical device can include one or more of the receiver-stimulators 110 of FIG. 1 and the tissue 206 can be cardiac tissue of the patient, such as endocardial tissue of the left ventricle.

As shown in FIG. 2A, the delivery system 240 can include an elongate sheath 242 (which can also be referred to as a shaft, a catheter, an elongate member, and the like). The sheath 242 defines a lumen 245 and has a distal portion 243 defining a distal opening 244 to the lumen 245. The sheath 242 is shown as transparent in FIGS. 2A and 2B for clarity. In the illustrated embodiment, the receiver-stimulator 110 is positioned within the sheath 242 and includes a body 212 having a distal portion 213 and proximal portion 215. The receiver-stimulator 110 can include an anchor mechanism 214, such as a barb or other structure configured to be inserted at least partially within the tissue 206 to secure the receiver-stimulator 110 to the tissue 206. In some embodiments, the anchor mechanism 214 comprises and/or includes a cathode (not shown; e.g., a stimulation electrode) and the body 212 comprises and/or includes an anode (not shown) for stimulating the tissue 206. The anchor mechanism 214 is configured to electrically contact the tissue 206 after and/or during delivery. In some embodiments, the receiver-stimulator 110 can include some features that are at least generally similar in structure and function, or identical in structure and function, to those of the receiver-stimulators disclosed in (i) U.S. Pat. No. 9,283,392, filed Sep. 24, 2010, and titled “TEMPORARY ELECTRODE CONNECTION FOR WIRELESS PACING SYSTEMS,” (ii) U.S. patent application Ser. No. 17/494,252, filed Aug. 17, 2021, and titled “IMPLANTABLE STIMULATION ASSEMBLIES HAVING TISSUE ENGAGEMENT MECHANISMS, AND ASSOCIATED SYSTEMS AND METHODS,” and/or (iii) U.S. patent application Ser. No. 17/522,662, filed Nov. 9, 2021, and titled “SYSTEMS AND METHODS FOR WIRELESS ENDOCARDIAL STIMULATION OF THE LEFT VENTRICULAR SEPTAL WALL,” each of which is incorporated herein by reference in its entirety.

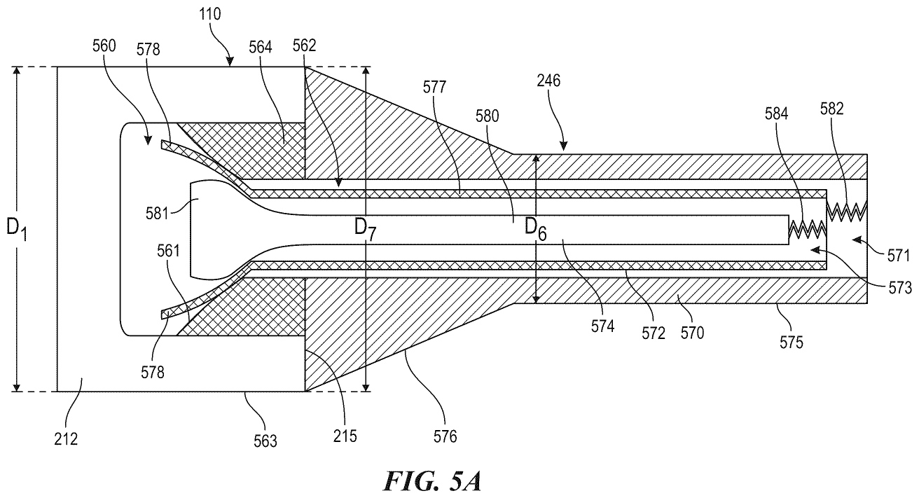

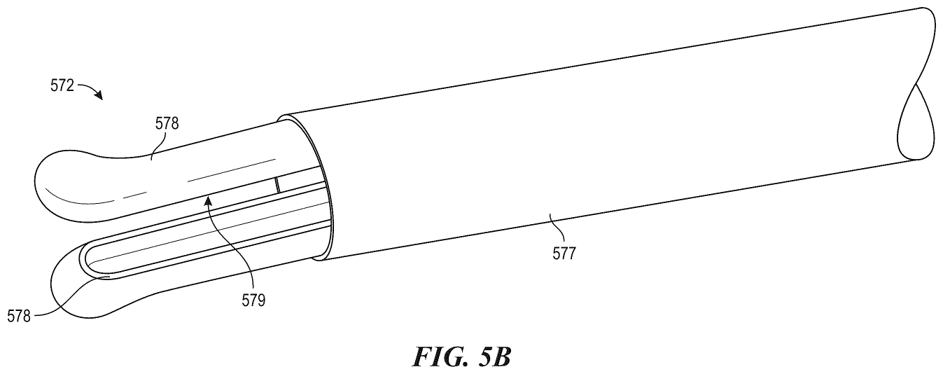

In some embodiments, the receiver-stimulator 110 (e.g., the proximal portion 215) is temporarily coupled to an attachment and advancement mechanism 246. The attachment and advancement mechanism 246 can be pushed distally and/or pulled proximally to advance/retract the receiver-stimulator 110 relative to the sheath 242, and can be actuated to release the receiver-stimulator 110. For example, the attachment and advancement mechanism 246 can be pushed distally to advance the receiver-stimulator 110 through the opening 244 into contact and/or engagement with the tissue 206 (e.g., to deploy/implant the receiver-stimulator 110) before being actuated to release the receiver-stimulator 110 in the tissue 206. Further details of the attachment and advancement mechanism 246 are described in detail below with reference to FIGS. 5A and 5B.

In some embodiments, during delivery, the receiver-stimulator 110 is temporarily electrically coupled to an external monitor and pacing controller via one or more conductive lines (not shown) routed through the sheath 242 and/or the attachment and advancement mechanism 246 to allow for externally controlled monitoring and pacing. In some embodiments, the delivery system 240 can include some features that are at least generally similar in structure and function, or identical in structure and function, to those of the delivery systems disclosed in U.S. Pat. No. 9,283,392, filed Sep. 24, 2010, and titled “TEMPORARY ELECTRODE CONNECTION FOR WIRELESS PACING SYSTEMS,” which is incorporated herein by reference in its entirety.

In the illustrated embodiment, the delivery system 240 further includes an elongate optical component 250 positioned within the sheath 242 and having a distal end portion 252. The optical component 250 can include, for example, one or more optical fibers (e.g., fiber optic cables) that can receive light through the distal end portion 252 and transmit/route the light to one or more image sensors configured to generate image data (e.g., images). The image data can be displayed on an external monitor or other device for viewing. The image sensors can be positioned proximal to the distal end portion 252 of the optical component 250 within the sheath 242 or external to the sheath 242. In some embodiments, the optical component 250 can include one or more lenses, mirrors, and/or other optical components at the distal end portion 252 or elsewhere and configured to focus, collect, or otherwise manipulate light received through the distal end portion 252. In some embodiments, the optical component 250 includes a light source configured to emit light from the distal end portion 252 into the sheath 242.

In the illustrated embodiment, the receiver-stimulator 110 has a first diameter D₁, the attachment and advancement mechanism 246 has a second diameter D₂ smaller than the first diameter D₁, the optical component has a third diameter D₃ smaller than the first diameter D₁ and greater than the second diameter D₂, and the sheath 242 has a fourth diameter D₄ greater than the first through third diameters D₁₋₃. In some embodiments, the fourth diameter D₄ can be about 12 French. In the first configuration shown in FIG. 2A, the reduced second diameter D₂ provides allows the optical component 250 to be positioned adjacent to the attachment and advancement mechanism 246 when packed within the sheath 242 such that the distal end portion 252 of optical component 250 is positioned behind the receiver-stimulator 110. This allows the sheath 242 to maintain the fourth diameter D₄ whether or not the optical component 250 is provided in the delivery system 240, thereby facilitating low profile delivery. In some aspects of the present technology, this allows the fourth diameter D₄ of the sheath 242 to be minimized. In some embodiments, the delivery system 240 is advanced through the vasculature of the patient to the tissue 206 in the first configuration. For example, the delivery system 240 can be inserted and advanced through an introducer sheath that penetrates the skin of the patient and extends partially into the vasculature of the patient.

Referring to FIGS. 2A and 2B together, the delivery system 240 can be moved from the first configuration (FIG. 2A) to the second configuration (FIG. 2B) by advancing the optical component 250 distally through the sheath 242 relative to the receiver-stimulator 110 and/or retracting the receiver-stimulator 110 proximally (e.g., via the attachment and advancement mechanism 246) through the sheath 242 relative to the optical component 250. Referring to FIG. 2B, in the second configuration, the optical component 250 is positioned at least partially adjacent the receiver-stimulator 110 such that the distal end portion 252 of the optical component 250 has an at least partially unobstructed view out of the opening 244 of the sheath 242. Accordingly, in the second configuration, the optical component 250 is positioned to facilitate direct visualization (e.g., image capture) of the tissue 206 proximate the opening 244 (e.g., out of the opening 244).

In some embodiments, advancing the optical component 250 to the second configuration causes the distal portion 243 of the sheath 242 to expand in cross-sectional area. For example, in the second configuration the distal portion 243 of the sheath 242 may take on a fifth diameter D₅ larger than the fourth diameter D₄. To allow for such an increase in cross-sectional area, the sheath 242 can be made from a compliant material such that the movement of the optical component 250 past the body 212 of the receiver-stimulator 110 expands the diameter of the sheath 242. In some embodiments, the optical component 250 is steerable and/or deflectable such that the optical component 250 can be steered and/or deflected past the proximal portion 215 of the body 212 of the receiver-stimulator 110. In some embodiments, the receiver-stimulator 110 and/or the attachment and advancement mechanism 246 is configured (e.g., shaped, sized) to facilitate the advancement of the optical component 250 past the proximal portion 215 of the body 212 as described, for example, in further detail below with reference to FIG. 5A.

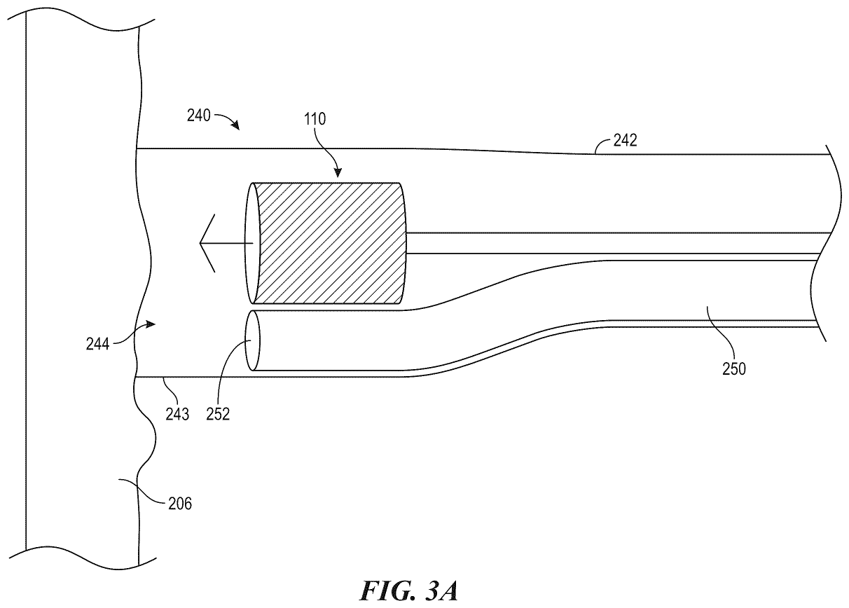

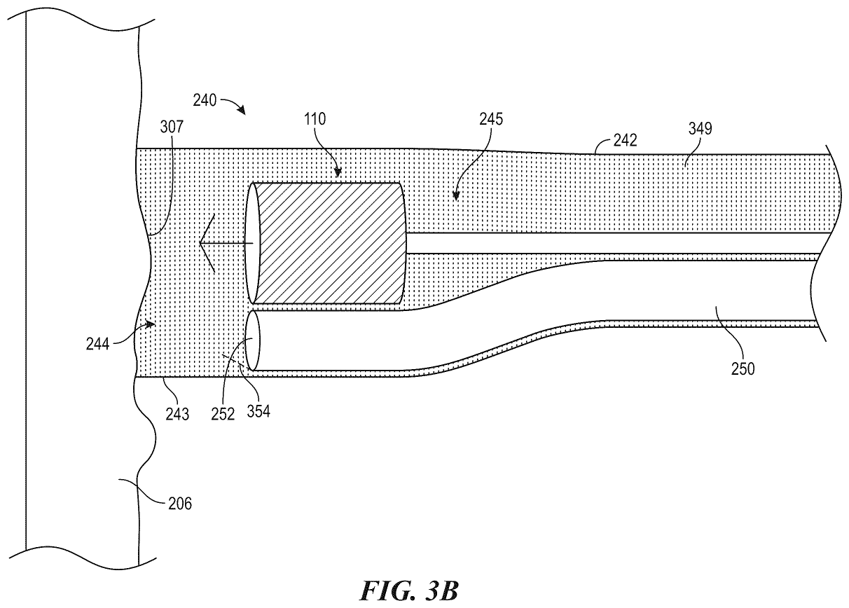

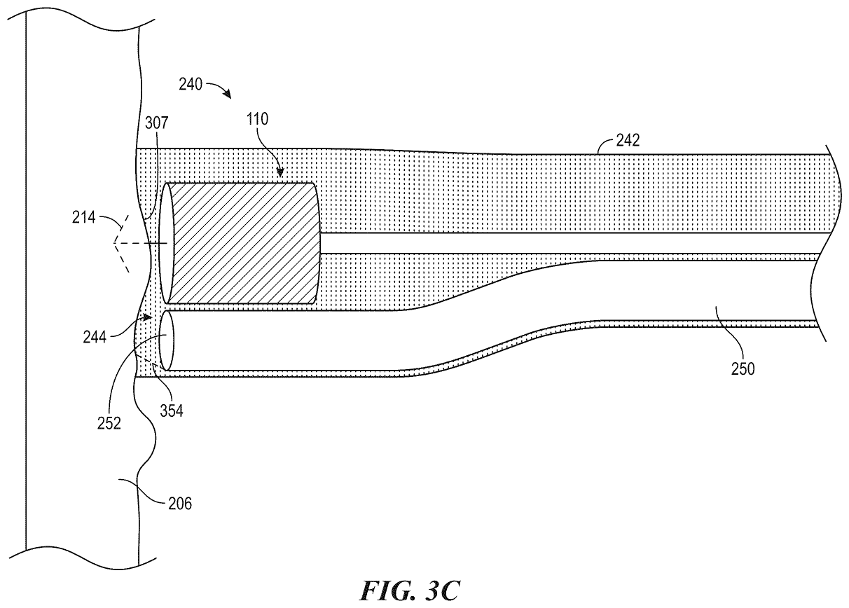

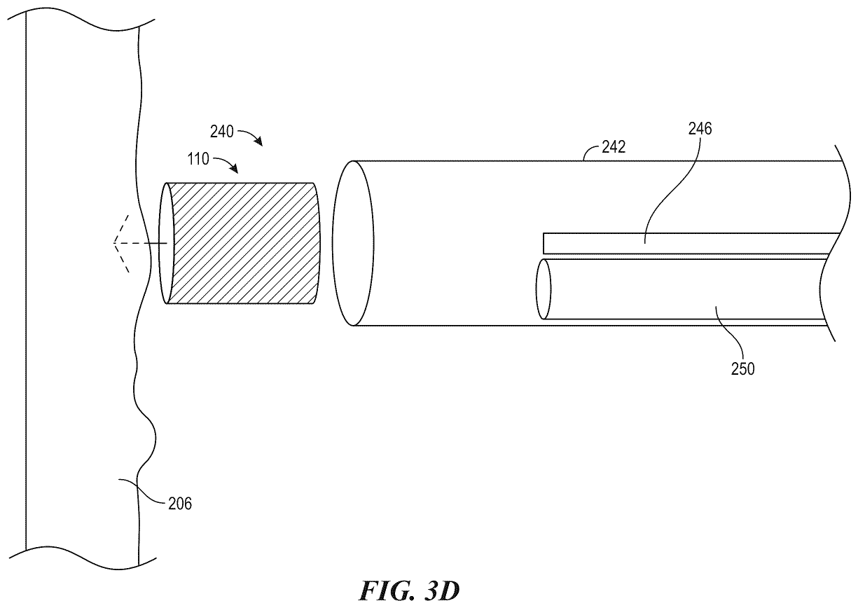

FIGS. 3A-3D are enlarged side views of the distal portion of the delivery system 240 during a procedure to implant the receiver-stimulator 110 in the tissue 206 in accordance with embodiments of the present technology. Referring first to FIG. 3A, the delivery system 240 can be advanced into contact with the tissue 206 in the second configuration in which the distal end portion 252 of the optical component 250 is positioned to at least partially adjacent to the receiver-stimulator 110 to facilitate viewing and imaging of the opening 244 of the sheath 242. Specifically, the distal portion 243 of the sheath 242 can be advanced into contact with the tissue 206 about a target location 307 of the tissue 206 for implantation of the receiver-stimulator 110 such that the opening 244 is adjacent the tissue 206. In other embodiments, the delivery system 240 can be advanced into contact with the tissue 206 in the first configuration (FIG. 2A) before being moved to the second configuration shown in FIG. 3A by advancing the optical component 250 relative to the receiver-stimulator 110 and/or retracting the receiver-stimulator 110 relative to the optical component 250.

Referring to FIG. 3B, the lumen 245 can be flushed with a fluid 349 as the distal end of the sheath 242 nears and comes in contact with the tissue 206 to evacuate blood and/or other material within the lumen 245 and/or directly in front of the opening 244. Accordingly, the portion of the lumen 245 forward of the optical component 250 and any space between the distal end of the sheath 242 and the optical component 250 is at least generally filled with the fluid 349. The fluid 349 can be an optically transparent or generally optically transparent fluid, such as saline, that facilitates a clear optical path between the distal end portion 252 of the optical component 250 and the tissue 206. A pressure of the fluid 349 in the lumen 245 can be greater than or the same as the pressure outside the sheath 242 (e.g., the blood pressure of the patient) such that the fluid 349 is maintained within the lumen 245 and the ingress of blood is inhibited. In some embodiments, the distal portion 243 of the sheath 242 can at least partially seal against the tissue 206 to help maintain the fluid 349 within the lumen 245. With the fluid 349 in the lumen 245, the optical component 250 can be used to visualize the target location 307 of the tissue 206 for implantation of the receiver-stimulator 110.

Optionally, in some embodiments the optical component 250 includes a mirror 354 and/or other reflective component/member oriented to help facilitate visualization of the target location 307. Specifically, the mirror 354 can reflect light into the distal end portion 252 of the optical component 250 that may not otherwise be within the field-of-view of the distal end portion 252. In some embodiments, the optical component 250 can be torqued to move the mirror 354 to provide different views within and/or out of the lumen 245. That is, the mirror 354 can make the view captured by the optical component 250 steerable. Alternatively or additionally, the optical component 250 can include one or more lenses or other components that facilitate a wide field-of-view from the distal end portion 252 that encompasses the target location 307.

In some embodiments, a user (e.g., a physician) of the delivery system 240 can evaluate the anatomy of the target location 307 based on the images from the optical component 250. For example, cardiac tissue is not typically smooth, but rather has a complex topology including fibrous and conductive structures, especially in diseased hearts. The user can confirm or deny that the target location 307 is suitable for implantation of the receiver-stimulator 110 based on the visualization of this structure of the target location 307. Specifically, the optical component 250 allows the user to view the target location 307 including the fibrous nature of the target location 307. The optical component 250 can also provide for visualization of visually-apparent conductive structures positioned at the target location 307, such as the left bundle branch (LBB) that runs along an outer surface of the endocardium and/or any muscular structures that may be suitable for electrical stimulation, such as the papillary muscles.

Referring to FIG. 3C, after confirming the target location 307, the receiver-stimulator 110 can be implanted within the tissue 206 by advancing the receiver-stimulator 110 distally through the sheath 242 and out of the opening 244 to secure the anchor mechanism 214 of the receiver-stimulator 110 within the tissue 206. In some embodiments, the optical component 250 is also advanced distally (e.g., together with the receiver-stimulator 110) to facilitate visualization of the securement of the receiver-stimulator 110 to the tissue 206 at the target location 307. For example, the user can confirm or deny that the receiver-stimulator 110 is sufficiently secured to the tissue 206 based on the images captured by the optical component 250.

If the receiver-stimulator 110 is not sufficiently secured, the receiver-stimulator 110 can be withdrawn from the tissue 206 for reimplantation at the same target location 307 or a different target location. Referring to FIG. 3D, if the receiver-stimulator 110 is sufficiently secured, the receiver-stimulator 110 can be detached from the attachment and advancement mechanism 246, and the sheath 242 and the optical component 250 can be withdrawn from the patient.

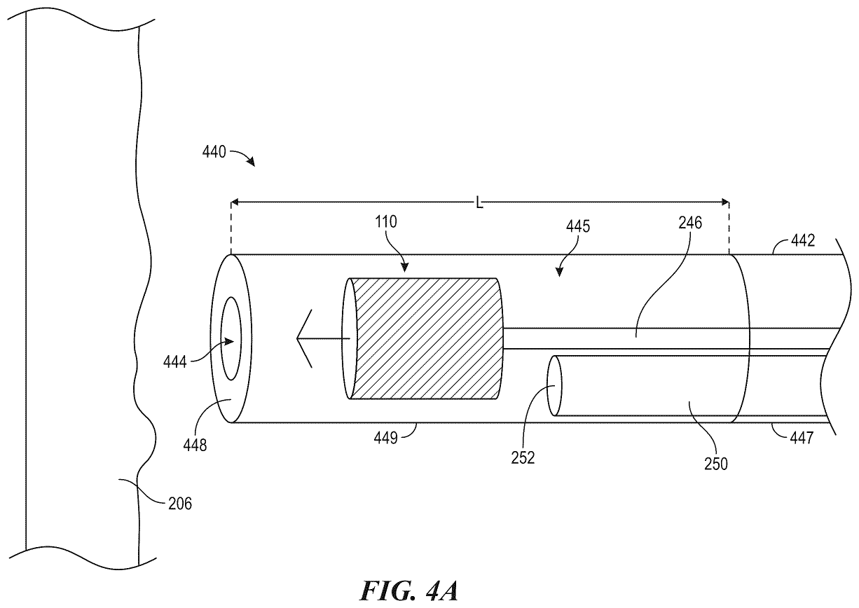

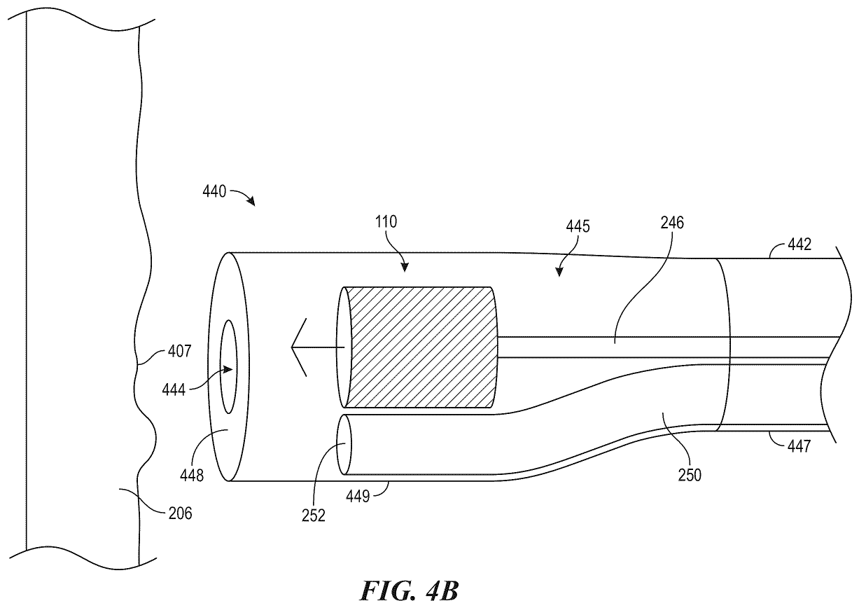

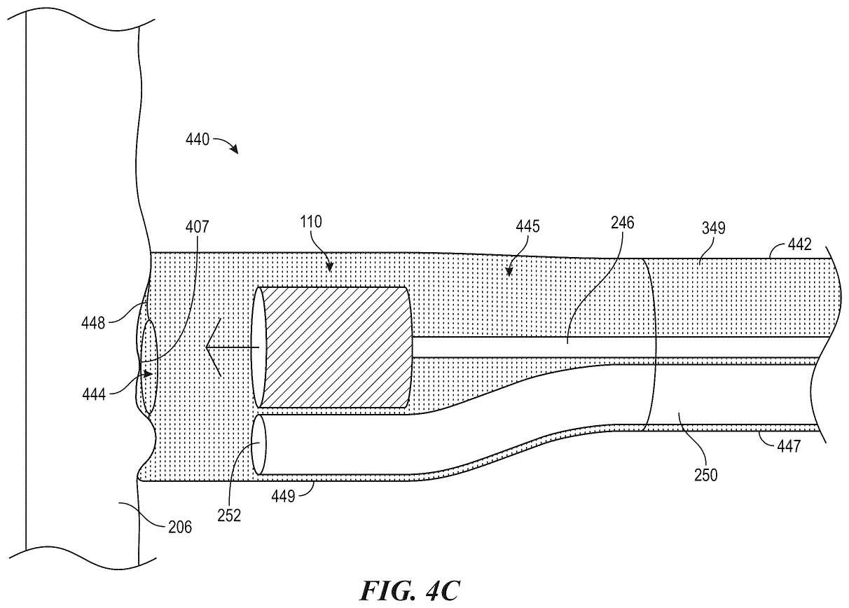

FIGS. 4A-4C are enlarged side views of a distal portion of a delivery system 440 configured to facilitate delivery of an implantable medical device, such as the receiver-stimulator 110 of FIGS. 2A-3D, to the tissue 206 in accordance with additional embodiments of the present technology. The delivery system 440 is in a first position or configuration (e.g., a delivery configuration, an advancement configuration) in FIG. 4A, and in a second position or configuration (e.g., a visualization configuration, an implantation configuration) in FIGS. 4B and 4C.

In some embodiments, the delivery system 440 includes some features that are at least generally similar in structure and function, or identical in structure and function, to the corresponding features of the delivery system 240 described in detail above with reference to FIGS. 2A-3D, and can operate in a generally similar or identical manner to the delivery system 240. For example, in the illustrated embodiment the delivery system 440 includes an elongate sheath 442 defining a lumen 445, and the receiver-stimulator 110, the attachment and advancement mechanism 246, and the optical component 250 are positioned within the lumen 445. The sheath 442 is shown as transparent in FIGS. 4A-4C for clarity.

In the illustrated embodiment, the sheath 442 includes a proximal portion 447 and a distal portion 449 (which can also be referred to as a distal hood, a distal element, a distal tip, and/or the like) extending from the proximal portion 447. The distal portion 449 defines a distal opening 444 to the lumen 445. The distal portion 449 can be formed of an optically transparent material (e.g., silicone) and can be more compliant, less rigid, and/or more flexible than the proximal portion 447, which can be formed of typical catheter materials. Accordingly, the distal portion 449 can be formed of a different material and/or a material having different rigidity, optical, and/or other characteristics than the proximal portion 447. In some embodiments, the distal portion 449 comprises a balloon and/or other structure having low radial strength. In the illustrated embodiment, the distal portion 449 further defines a distal ring 448 about the opening 444. The ring 448 can be configured (e.g., shaped, sized, formed of compliant material) to be atraumatic and define an atraumatic tip of the sheath 442. In some embodiments, the distal portion 449 has a length L of between about 15-50 millimeters (e.g., between about 30-40 millimeters).

In the first configuration shown in FIG. 4A, the optical component 250 is packed within the sheath 442 behind the receiver-stimulator 110. In the illustrated embodiment, the receiver-stimulator 110 and the distal end portion 252 of the optical component 250 are positioned in the distal portion 449 of the sheath 442. In other embodiments, the optical component 250, or the both optical component 250 and the receiver-stimulator 110, can be retracted farther proximally within the sheath 442 in the first configuration. For example, the distal end portion 252 of the optical component 250 and the receiver-stimulator 110 can be positioned proximally of the distal portion 449 of the sheath 442 in the first configuration (e.g., such as in an intermediate or proximal portion of the sheath 442).

In the second configuration shown in FIGS. 4B and 4C, the optical component 250 is positioned at least partially adjacent the receiver-stimulator 110 such that the distal end portion 252 of the optical component 250 has an at least partially unobstructed view out of the opening 444 and/or proximate the opening 444. Referring to FIGS. 4B and 4C together, the delivery system 440 can be moved to the second configuration (FIG. 4B) and then advanced into contact with the tissue 206 (FIG. 4C) about a target location 407 of the tissue 206 for implantation of the receiver-stimulator 110. The delivery system 440 can be moved from the first configuration (FIG. 4A) to the second configuration (FIGS. 4B and 4C) by advancing the optical component 250 distally through the sheath 442 relative to the receiver-stimulator 110 and/or retracting the receiver-stimulator 110 proximally (e.g., via the attachment and advancement mechanism 246) through the sheath 442 relative to the optical component 250.

In the second configuration, the distal portion 449 of the sheath 442 is radially expanded. The optical component 250 can provide for direct visualization of the tissue 206 at and/or proximate the target location 407 through the opening 444 and/or through the distal portion 449 (e.g., through the ring 448), which can be formed of an optically transparent material. As described in detail above, such visualization can enable a user to evaluate the anatomy of the target location 407 and/or a sufficiency of anchoring of the receiver-stimulator 110 at the target location 407.

Referring to FIG. 4C, the ring 448 can engage the tissue 206 about the target location 407 to at least partially seal the lumen 445 of the sheath 442. In some aspects of the present technology, the distal portion 449 is formed of a compliant material such that the ring 448 can engage and seal against (e.g., conform to) the complex topology of the tissue 206. The lumen 445 can be flushed with the fluid 349 to evacuate blood and/or other material within the lumen 445 out of the opening 444 such that the lumen 445 is at least generally filled with the fluid 349. In some embodiments, the distal portion 449 is radially expanded (e.g., inflated) via the pressure of the fluid 349 within the lumen 445. With the fluid 349 filling the lumen 445, the anatomy of the target location 407 and the expansion of the distal portion 449 can be directly visualized via the optical component 250, and the receiver-stimulator 110 can be advanced into and secured to the tissue 206.

In other embodiments, the sheath 442 can additionally or alternatively include multiple optical sensors (e.g., cameras) positioned within the distal portion 449, such as at and/or within the ring 448. The optical sensors can each have a field-of-view oriented to view the target location 407, and can each transmit signals through wires and/or other components positioned in the wall of the sheath 442.

FIG. 5A is an enlarged side cross-sectional view of a portion of the receiver-stimulator 110 and the attachment and advancement mechanism 246 shown in FIG. 2A in accordance with embodiments of the present technology. In the illustrated embodiment, the body 212 of the receiver-stimulator 110 includes an outer surface 563 an inner surface 561 defining a proximal cavity 560 having an opening 562. In some embodiments, the receiver-stimulator 110 can further comprise an insulator 564 (e.g., a feedthrough insulator) that electrically isolates the inner surface 561 from the outer surface 563 of the body 212. The attachment and advancement mechanism 246 can comprise an elongate outer member 570 (e.g., a pusher tube, an outer tube, an outer elongate member, and/or the like), an elongate intermediate member 572 (e.g., a finger tube, an intermediate tube, an intermediate elongate member, and/or the like), and an elongate inner member 574 (e.g., a ball wire, an inner wire, an inner elongate member, and/or the like). The outer member 570 can define a first lumen 571, and the intermediate member 572 can be positioned at least partially within the first lumen 571. The intermediate member 572 can define a second lumen 573, and the inner member 574 can be positioned at least partially within the second lumen 573.

In the illustrated embodiment, the outer member 570 includes an outer surface 575 having a tapered surface portion 576 proximate the receiver-stimulator 110. In some embodiments, a diameter of the of the outer surface 575 can increase from a sixth diameter D₆ to a seventh diameter D₇ along the tapered surface portion 576. In some embodiments, the seventh diameter D₇ is the same as or about the same as the first diameter D₁ of the body 212 of the receiver-stimulator 110. Referring to FIGS. 2A-5A together, in some aspects of the present technology, the tapered surface portion 576 can facilitate the advancement of the optical component 250 past the receiver-stimulator 110 when the delivery system 240 (FIGS. 2A-3D) and/or the delivery system 440 (FIGS. 4A-4C) are moved from the first configuration to the second configuration. For example, as the optical component 250 is advanced and/or the receiver-stimulator 110 is retracted, the tapered surface portion 576 can engage the distal end portion 252 of the optical component 250 to deflect the optical component 250 radially outward away from the body 212 of the receiver-stimulator 110 such that the optical component 250 does not snag or otherwise engage the proximal portion 215 of the body 212. Such radially outward movement of the optical component 250 can also act to expand the distal portion 243 of the sheath 242 (FIGS. 2A-3D) and/or the distal portion 449 of the sheath 442 (FIGS. 4A-4C).

Referring again to FIG. 5A, in the illustrated embodiment the intermediate member 572 includes a proximal portion 577 and one or more (e.g., a pair) of distal fingers 578 extending from the proximal portion 577. FIG. 5B is an enlarged perspective view of a distal portion of the intermediate member 572 in accordance with embodiments of the present technology. As best seen in FIG. 5B, the distal fingers 578 can be separated by one or more corresponding slots 579. The distal fingers 578 are configured to flex radially outward relative to the proximal portion 577 within the cavity 560 as described in detail below.

Referring to FIG. 5A, the inner member 574 includes a proximal portion 580 and an enlarged distal portion 581 extending from the proximal portion 580. In some embodiments, the inner member 574 is a ball wire in which the proximal portion 580 comprises a wire and the enlarged distal portion 581 comprises a ball coupled to the wire. The enlarged distal portion 581 can have a diameter sized to fit through the opening 562 in the body 212 of the receiver-stimulator 110.

In the illustrated embodiment, the attachment and advancement mechanism 246 further includes (i) a first biasing member 582 operably coupled between the outer member 570 and the intermediate member 572 and (ii) a second biasing member 584 operably coupled between the intermediate member 572 and the inner member 574. The first and second biasing members 582, 584 can be springs and/or other members that exert a force between the outer member 570, the intermediate member 572, and the inner member 574 as described herein.

In the illustrated embodiment, the attachment and advancement mechanism 246 is secured to the receiver-stimulator 110 in a deployment configuration. Specifically, the enlarged distal portion 581 of the inner member 574 is positioned within the cavity 560 of the receiver-stimulator 110 and is pulled proximally against the distal fingers 578 of the intermediate member 572 by the second biasing member 584. The engagement of the enlarged distal portion 581 with the distal fingers 578 radially expands the distal fingers 578 within the proximal cavity 560 to secure the attachment and advancement mechanism 246 therewithin. For example, the distal fingers 578 can engage the inner surface 561 of the cavity 560 and have an expanded diameter that is too large to be pulled through the opening 562 in the body 212 of the receiver-stimulator 110.

Further, in the deployment configuration shown in FIG. 5A, the first biasing member 582 is configured (e.g., shaped, positioned) to pull the intermediate member 572 proximally relative to the outer member 570. This force acts to pull the body 212 of the receiver-stimulator 110 toward and/or into contact with the outer member 570 such that, for example, the receiver-stimulator 110 and the outer member 570 are stabilized against one another. In some aspects of the present technology, such stabilization can improve the pushability of the attachment and advancement mechanism 246 and the receiver-stimulator 110 together, such as during deployment and implantation of the receiver-stimulator 110.

To release the attachment and advancement mechanism 246 from the receiver-stimulator 110, the inner member 574 can be advanced distally relative to the intermediate member 572 against the biasing force of the second biasing member 584 to move the enlarged distal portion 581 distally through the cavity 560 and away from the distal fingers 578. When the enlarged distal portion 581 is moved away from the distal fingers 578, the biasing force of the first biasing member 582 pulls the distal fingers 578 proximally against the inner surface 561 such that the fingers collapse and are pulled through the opening 562. After and/or while the distal fingers 578 collapse and exit the opening 562, the biasing force of the second biasing member 584 can pull the inner member 574 through the opening 562—thereby detaching the attachment and advancement mechanism 246 from the receiver-stimulator 110.

Referring to FIGS. 2A-4C together, in some embodiments the optical component 250 can be exchangeable with additional components. For example, during advancement of the delivery system 240/440 through the vasculature of a patient, the delivery system 240/440 can include a catheter (e.g., a pigtail catheter) and/or a guidewire in place of the optical component 250. Such components can help facilitate advancement of the delivery system 240/440 to the tissue 206. For example, a pigtail catheter can be used to facilitate traversal across a heart valve and into a ventricle where the tissue 206 is located. After advancement, the catheter and/or other components can be removed and exchanged for the optical component 250. Similarly, in some embodiments the receiver-stimulator 110, the attachment and advancement mechanism 246, and the optical component 250 can be integrated into a single exchangeable catheter system that can be exchanged for other components used to facilitate delivery of the delivery system 240/440 to proximate the tissue 206.

### IV. Additional Examples

The following examples are illustrative of several embodiments of the present technology:
1. A system for delivering an electrical stimulation implant, comprising:
an elongate sheath having a distal portion with a distal opening;
an attachment mechanism disposed within the sheath and configured to be releasably coupled to the electrical stimulation implant; and
an elongate optical component movably positioned within the sheath, wherein the optical component is configured to capture image data, wherein the optical component is movable from a first configuration to a second configuration, and wherein—
   in the first configuration, the optical component is positioned proximal to the electrical stimulation implant within the sheath,
   in the second configuration, the optical component is positioned at least partially adjacent to the electrical stimulation implant to capture image data of a region at and/or near the distal opening, and
   the distal portion of the elongate sheath is configured to expand in cross-sectional dimension when the optical component moves from the first configuration to the second configuration.
2. The system of example 1 wherein the distal portion comprises a compliant material.
3. The system of example 2 wherein the compliant material is optically transparent.
4. The system of example 2 or example 3 wherein the sheath has a proximal portion extending from the distal portion, and wherein the proximal portion comprises a different material than the compliant material.
5. The system of any one of examples 1-4 wherein the attachment mechanism comprises an outer member having a distal portion with a tapered surface, wherein the tapered surface is configured to deflect the optical component radially outward past the electrical stimulation implant when the optical component moves from the first configuration to the second configuration.
6. The system of any one of examples 1-5, further comprising the electrical stimulation implant configured to receive acoustic energy from a separate controller-transmitter and convert the acoustic energy to electrical stimulation energy.
7. The system of any one of examples 1-6 wherein the optical component comprises a distal end portion and a reflective component coupled to distal end portion, wherein the reflective component is configured to reflect light into the distal end portion of the optical component.
8. The system of any one of examples 1-7 wherein the optical component further comprises a light source configured to emit light through the distal opening of the sheath when the optical component is in the second configuration.
9. A method of intravascularly delivering an electrical stimulation implant to cardiac tissue of a heart of the patient, the method comprising:
positioning an elongate sheath such that a distal end portion of the sheath is in contact with the cardiac tissue;
positioning the electrical stimulation implant within a lumen of the sheath;
positioning an optical component within the lumen;
moving the optical component from (a) a first position in which the optical component is positioned proximal to the electrical stimulation implant within the lumen to (b) a second position in which the optical component is positioned at least partially adjacent to the electrical stimulation implant within the lumen; and
capturing, via the optical component, image data of the cardiac tissue proximate to the distal end portion of the sheath with the optical component in the second position.
10. The method of example 9 wherein moving the optical component from the first position to the second position comprises at least partially expanding the distal end portion of the sheath.
11. The method of example 10 wherein the distal end portion of the sheath comprises a compliant material.
12. The method of any one of examples 9-11 wherein the method further comprises at least generally filling the lumen with an optically-transparent fluid, and wherein capturing the image data comprises capturing the image data with the optically-transparent fluid within the lumen.
13. The method of any one of examples 9-12 wherein the method further comprises:
evaluating the issue proximate the distal end portion of the sheath based on the captured image data;
determining that the cardiac tissue proximate the distal end portion of the sheath is suitable for implantation of the electrical stimulation component; and
securing the electrical stimulation implant to the cardiac tissue.
14. The method of any one of examples 9-13 wherein the method further comprises:
evaluating the cardiac issue proximate the distal end portion of the sheath based on the captured image data;
determining that the cardiac tissue proximate the distal end portion of the sheath is suitable for implantation of the electrical stimulation component; and
securing the electrical stimulation implant to the cardiac tissue.
15. The method of any one of examples 9-14 wherein the image data includes image data of a conductive structure of the heart.
16. The method of any one of examples 9-15 wherein the method further comprises:
evaluating the cardiac issue proximate the distal end portion of the sheath based on the captured image data;
determining that the cardiac tissue proximate the distal end portion of the sheath is suitable or not suitable for implantation of the electrical stimulation component;
if the cardiac tissue proximate the distal end portion of the sheath is suitable for implantation, securing the electrical stimulation implant to the cardiac tissue; and
if the cardiac tissue proximate the distal end portion of the sheath is not suitable for implantation, repositioning the sheath such that the distal end portion of the sheath is in contact with a different region of the cardiac tissue.
17. A system, comprising:
an elongate sheath having a distal portion with a distal opening;
an electrical stimulation implant disposed within the sheath, wherein the electrical stimulation implant includes a proximal portion defining a cavity and a proximal opening to cavity;
an attachment mechanism disposed within the sheath and configured to be releasably secured to the electrical stimulation implant, wherein the attachment mechanism comprises—
   an elongate outer member;
   an elongate intermediate member extending through the elongate outer member, wherein the intermediate member includes at least one finger biased radially inward; and
   an elongate inner member extending through the intermediate member, wherein the inner member includes an enlarged distal portion;
   wherein, in a secured position, the intermediate member extends through the proximal opening such that the at least one finger is positioned within the cavity and the enlarged portion of the inner member deflects the at least one finger radially outward to engage the electrical stimulation implant and secure the electrical stimulation implant to the attachment mechanism;
   wherein distal movement of the inner member permits the at least one finger to move radially inward to disengage the electrical stimulation implant such that the attachment mechanism is detached from the electrical stimulation implant; and
an elongate optical component movably positioned within the sheath, wherein the optical component is configured to capture image data of a region at and/or near the distal opening.
18. The system of example 17 wherein the optical component is movable from a first configuration to a second configuration, and wherein—
   in the first configuration, the optical component is positioned proximal to the electrical stimulation implant within the sheath,
   in the second configuration, the optical component is positioned at least partially adjacent to the electrical stimulation implant to capture the image data of the region at and/or near the distal opening, and
   the distal portion of the elongate sheath is configured to expand in cross-sectional dimension when the optical component moves from the first configuration to the second configuration.
19. The system of example 17 or example 18 wherein the outer member has a distal portion with a tapered surface, wherein the tapered surface is configured to deflect the optical component radially outward past the electrical stimulation implant when the optical component is moved from the first configuration to the second configuration.
20. The system of any one of examples 17-19 wherein the electrical stimulation implant is configured to receive acoustic energy from a separate controller-transmitter and convert the acoustic energy to electrical stimulation energy.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for delivering an electrical stimulation implant, comprising:
an elongate sheath having a distal portion with a distal opening;
an attachment mechanism disposed within the sheath and configured to be releasably coupled to the electrical stimulation implant; and
an elongate optical component movably positioned within the sheath, wherein the optical component is configured to capture image data, wherein the optical component is movable from a first configuration to a second configuration, and wherein—
in the first configuration, the optical component is positioned proximal to the electrical stimulation implant within the sheath,
in the second configuration, the optical component is positioned at least partially adjacent to the electrical stimulation implant to capture image data of a region at and/or near the distal opening, and
the distal portion of the elongate sheath is configured to expand in cross-sectional dimension when the optical component moves from the first configuration to the second configuration.

2. The system of claim 1 wherein the distal portion comprises a compliant material.

3. The system of claim 2 wherein the compliant material is optically transparent.

4. The system of claim 2 wherein the sheath has a proximal portion extending from the distal portion, and wherein the proximal portion comprises a different material than the compliant material.

5. The system of claim 1 wherein the attachment mechanism comprises an outer member having a distal portion with a tapered surface, wherein the tapered surface is configured to deflect the optical component radially outward past the electrical stimulation implant when the optical component moves from the first configuration to the second configuration.

6. The system of claim 1, further comprising the electrical stimulation implant configured to receive acoustic energy from a separate controller-transmitter and convert the acoustic energy to electrical stimulation energy.

7. The system of claim 1 wherein the optical component comprises a distal end portion and a reflective component coupled to distal end portion, wherein the reflective component is configured to reflect light into the distal end portion of the optical component.

8. The system of claim 1 wherein the optical component further comprises a light source configured to emit light through the distal opening of the sheath when the optical component is in the second configuration.

9. A method of intravascularly delivering an electrical stimulation implant to cardiac tissue of a heart of the patient, the method comprising:
positioning an elongate sheath such that a distal end portion of the sheath is in contact with the cardiac tissue;
positioning the electrical stimulation implant within a lumen of the sheath;
positioning an optical component within the lumen;
moving the optical component from (a) a first position in which the optical component is positioned proximal to the electrical stimulation implant within the lumen to (b) a second position in which the optical component is positioned at least partially adjacent to the electrical stimulation implant within the lumen; and
capturing, via the optical component, image data of the cardiac tissue proximate to the distal end portion of the sheath with the optical component in the second position.

10. The method of claim 9 wherein moving the optical component from the first position to the second position comprises at least partially expanding the distal end portion of the sheath.

11. The method of claim 10 wherein the distal end portion of the sheath comprises a compliant material.

12. The method of claim 9 wherein the method further comprises at least generally filling the lumen with an optically-transparent fluid, and wherein capturing the image data comprises capturing the image data with the optically-transparent fluid within the lumen.

13. The method of claim 9 wherein the method further comprises:
evaluating the issue proximate the distal end portion of the sheath based on the captured image data;
determining that the cardiac tissue proximate the distal end portion of the sheath is suitable for implantation of the electrical stimulation component; and
securing the electrical stimulation implant to the cardiac tissue.

14. The method of claim 9 wherein the method further comprises:
evaluating the cardiac issue proximate the distal end portion of the sheath based on the captured image data;
determining that the cardiac tissue proximate the distal end portion of the sheath is suitable for implantation of the electrical stimulation component; and
securing the electrical stimulation implant to the cardiac tissue.

15. The method of claim 9 wherein the image data includes image data of a conductive structure of the heart.

16. The method of claim 9 wherein the method further comprises:
evaluating the cardiac issue proximate the distal end portion of the sheath based on the captured image data;
determining that the cardiac tissue proximate the distal end portion of the sheath is suitable or not suitable for implantation of the electrical stimulation component;
if the cardiac tissue proximate the distal end portion of the sheath is suitable for implantation, securing the electrical stimulation implant to the cardiac tissue; and
if the cardiac tissue proximate the distal end portion of the sheath is not suitable for implantation, repositioning the sheath such that the distal end portion of the sheath is in contact with a different region of the cardiac tissue.

17. A system, comprising:
an elongate sheath having a distal portion with a distal opening;
an electrical stimulation implant disposed within the sheath, wherein the electrical stimulation implant includes a proximal portion defining a cavity and a proximal opening to cavity;
an attachment mechanism disposed within the sheath and configured to be releasably secured to the electrical stimulation implant, wherein the attachment mechanism comprises—
an elongate outer member;
an elongate intermediate member extending through the elongate outer member, wherein the intermediate member includes at least one finger biased radially inward; and
an elongate inner member extending through the intermediate member, wherein the inner member includes an enlarged distal portion;
wherein, in a secured position, the intermediate member extends through the proximal opening such that the at least one finger is positioned within the cavity and the enlarged portion of the inner member deflects the at least one finger radially outward to engage the electrical stimulation implant and secure the electrical stimulation implant to the attachment mechanism;
wherein distal movement of the inner member permits the at least one finger to move radially inward to disengage the electrical stimulation implant such that the attachment mechanism is detached from the electrical stimulation implant; and
an elongate optical component movably positioned within the sheath, wherein the optical component is configured to capture image data of a region at and/or near the distal opening.

18. The system of claim 17 wherein the optical component is movable from a first configuration to a second configuration, and wherein—
in the first configuration, the optical component is positioned proximal to the electrical stimulation implant within the sheath,
in the second configuration, the optical component is positioned at least partially adjacent to the electrical stimulation implant to capture the image data of the region at and/or near the distal opening, and
the distal portion of the elongate sheath is configured to expand in cross-sectional dimension when the optical component moves from the first configuration to the second configuration.

19. The system of claim 17 wherein the outer member has a distal portion with a tapered surface, wherein the tapered surface is configured to deflect the optical component radially outward past the electrical stimulation implant when the optical component is moved from the first configuration to the second configuration.

20. The system of claim 17 wherein the electrical stimulation implant is configured to receive acoustic energy from a separate controller-transmitter and convert the acoustic energy to electrical stimulation energy.
